(19) United States
(12) Patent Application Publication
(10) Pub. No.: US 2011/0218472 A1
(43) Pub. Date: Sep. 8, 2011

(54) NON DRUG BASED WOUND DRESSING POLYMER FILM AND A METHOD OF PRODUCING THE SAME

(75) Inventors: HAMID MIRZADEH, TEHRAN (IR); HAMID MAHDAVI, TEHRAN (IR)

(73) Assignee: IRAN POLYMER AND PETROCHEMICAL INSTITUTE (IPPI), TEHRAN (IR)

(21) Appl. No.: 12/898,796

(22) Filed: Oct. 6, 2010

Related U.S. Application Data
(60) Provisional application No. 61/309,454, filed on Mar. 2, 2010.

(51) Int. Cl. A61F 13/02

(52) U.S. Cl. 602/43; 427/2.31

(57) ABSTRACT

The various embodiments herein provide a polymer wound dresser in which no drug is used and a method of producing the same. According to an embodiment, a wound dressing film comprises a base polymer film and a laminated backing layer on the base polymer film. The base polymer film includes a natural polymer, a biopolymer and an artificial water absorber polymer. The natural polymer is a chitosan having a preset weight percentage. The biopolymer comprises one or more element selected from a group comprising of a gelatin having a preset weight percentage and a collagen having a preset weight percentage or a combination thereof. The artificial water absorber polymer is selected from a group comprising of a hydrophilic material having a preset weight percentage. The wound dressing film is a single film or a double layered film or a three layered film.

This application claims the benefit of Provisional Application No. 61/309,454, filed on Mar. 2, 2010.

### BACKGROUND

1. Technical Field

The embodiments herein generally relate to a field of medical treatment and particularly to wound dressings. The embodiments herein more particularly relates to wound dressers and a method of producing the same. The embodiments herein more particularly relates to a non-drug 4-element polymer films used as a wound dresser and method of producing the same.

2. Description of the Related Art

A dressing is an adjunct used by a person for application to a wound to promote healing and/or prevent further harm. A dressing is designed to be in direct contact with the wound and which makes it different from a bandage and is primarily used to hold a dressing in place.

A dressing can have a number of purposes depending on the type, severity and position of the wound, although all purposes are focused towards promoting recovery and preventing further harm from the wound. The Key purposes of the dressing are as follows:

1. Stem bleeding—helps to seal the wound to expedite the clotting process;
2. Absorb exudates—Soak up blood, plasma and other fluids exuded from the wound, containing it in one place;
3. Ease pain—Some dressings may have a pain relieving effect, and others may have a placebo effect;
4. Debride the wound—The removal of slough and foreign objects from the wound, Protection from infection and mechanical damage; and
5. Promote healing—through granulation and epithelialisation.

Modern dressings include gauzes (which may be impregnated with an agent designed to help sterility or to speed healing), films, gels, foams, hydrocolloids, alginates, hydrogels and polysaccharide pastes, granules and beads. Many gauze dressings have a layer of nonstick film over the absorbent gauze to prevent the wound from adhering to the dressing. Dressings can be impregnated with antiseptic chemicals, as in boracic lint or where medicinal Castor oil was used in the first surgical dressings.

In the 1960s, George Winter published his controversial research on moist healing. Previously, the accepted wisdom was that the wound should be kept as dry as possible to prevent an infection of a wound. Winter demonstrated that wounds kept moist healed faster than those exposed to the air or covered with traditional dressings.

Various types of dressings can be used to accomplish different objectives including the controlling of the moisture content so that the wound stays moist or dry, protecting the wound from infection, removing slough, and maintaining the optimum pH and temperature to encourage a healing.

The occlusive dressings, made from substances impervious to moisture such as plastic or latex, can be used to increase the rate of absorption of certain topical medications into the skin.

The modern dressings will almost all come in a prepackaged sterile wrapping, date coded to ensure sterility. This is because it will come in to direct contact with the wound and sterility is required to fulfill the ‘protection from infection’ which is an aim of a dressing.

Historically and still for cases in many less developed areas and in an emergency, the dressings are often improvised as needed. This can consist of anything including a clothing or a spare material, which will fulfill some of the basic tenets of a dressing such as stemming bleeding and absorbing exudates. Applying and changing dressings is one common task in nursing.

An “ideal” wound dressing is one that is sterile, breathable and conducive for a moist healing environment. These will then reduce the risk of infection, help to heal the wound more quickly and reduce generation of a scar.

Most of the advanced wound dressings are prepared from polymers which are natural or synthetics. Although natural polymers are biocompatible, their mechanical properties are weak. Unlike natural polymers, synthetic polymers have good mechanical properties, but their biocompatibility is lower in comparison with natural polymers.

Gelatin, collagen and chitosan are three natural materials which have many applications in wound dressings. Different studies show that the cells distinguish collagens specifically. The grown fibroblasts become flat on collagen and pseudo pods are attached to the surface of collagen. Mean while, it is observed that fibroblast body applies a pressure which leads to further orientation of the collagen mold and along with common biological degradation of collagen, it causes the formation of reconstructed molds. In addition to the above results, collagen adheres naturally to the wounds due to a binding to a fibrinogen. Long term adherence to the wound is necessary for controlling the growth of fibroblasts and preventing from the generation of scar tissues. Generally, the collagen reduces pain, limits infection and improves a healing process.

Gelatin is a colloidal protein and the oldest macromolecule which is obtained from the hydrolysis of collagen in skin, bone and the connective tissues of animals such as livestock, poultry and aquatics. It is one of the most used colloidal proteins in food, medicine, medical and military industries. It is also one of the biopolymers which have been used as wound dressings. This substance retains it capability for adhering to the collagen. The dressings containing gelatin, can be used without the need to thrombin or other additives.

Chitosan is a biodegradable, biocompatible and bio-adhesive polymer. This substance is antibacterial and antifungal and can prevent from the growth of bacteria which may cause infections. The microbial property of chitosan provides a protection against a wide range of microorganisms such as fungal, bacteria and viruses. This property of chitosan depends on these factors such as a type of a chitosan, a degree of polymerization, a chemical composition of food substrate, and environmental conditions such as the aqueous activation of the substrate or moisture or both of them. Thus, nowadays the application of this substance has a special place as a wound dressing in medical science.

Furthermore, many studies and researches have been done on it especially in most of the scientific and industrial circles. This substance is usually used as a gel or film for treatment of injuries and burns.

However, one of the main problems in using natural polymers such as chitosan, gelatin and collagen or may be a combination of them as a film will be their weak mechanical properties and their brittleness. Therefore, various methods have been developed in which these three followings could be mentioned: a) Combination with other polymers such as polyvinyl alcohol, cellulose and polyethylene oxide; b) Using chemical or physical cross-linking agents; c) Chemical synthesis of chitosan copolymers.

Due to the problems in the process of synthesizing and reversible properties that are obtained due to the physical cross-links, the combination of polymers with other materials is easy from the point of view of processing and very commodious from the point of view of economics.

Polyvinyl alcohol is a semi crystalline polymer which is soluble in water. This polymer has the chemical and thermal stability and excellent waster permeability. It is biologically compatible with human and degradable. That's why it is applied in medical science such as skin, cartilage, eye lens, vocal cords repairing and wound dressings. The specific properties of synthetic polymer such as PVA and biopolymers such as collagen, gelatin and chitosan can be combined to create new materials with unique structural, biological performance and mechanical properties appropriate for wound dressing.

Hence there is a need for a wound dresser with a highly water absorbing characteristics, a quick drying property, a highly bio-compatible property and mechanical properties, to act as a dam or barrier against an input and growth of microorganisms that prevents a wound from drying and scare. Also there is a need for a wound dresser having oxygen permeability, ability for sterilizing, the capability to stanch bleeding and adherence, good consistency with the skin anatomy, adequate resistance against tear, highly stable and durable in the applied temperature range.

The embodiments herein minimize and eliminate some of the above mentioned failures by utilizing a novel method and the structural features are described here.

The above mentioned shortcomings, disadvantages and problems are addressed herein and which will be understood by reading and studying the following specification.

### OBJECTIVES OF THE EMBODIMENTS

The primary object of the embodiments herein is to provide a non-drug based polymer film containing 4 elements as a wound dressing.

Another object of the embodiments herein is to provide a wound dressing designed with a combination of synthetic polymers and natural polymers.

Yet another object of the embodiments herein is to provide a method of producing a non-drug based modern polymer film as a wound dressing.

Yet another object of the embodiments herein is to provide a wound dressing with uniform thickness and good repeatability.

Yet another object of the embodiments herein is to provide a wound dressing with highly absorbent properties.

Yet another object of the embodiments herein is to provide a wound dressing having a high biocompatibility and mechanical properties.

Yet another object of the embodiments herein is to provide a wound dressing that prevents the wound from drying and scaring.

Yet another object of the embodiments herein is to provide a wound dressing with high oxygen permeability property.

Yet another object of the embodiments herein is to provide a wound dressing having the ability for sterilizing.

Yet another object of the embodiments herein is to provide a wound dressing having the capability to stanch bleeding and adherence.

Yet another object of the embodiments herein is to provide a wound dressing having a good consistency with the skin anatomy.

Yet another object of the embodiments herein is to provide a wound dressing having an adequate resistance against wear and tear.

Yet another object of the embodiments herein is to provide a wound dressing which is highly stable in the applied range of temperature.

Yet another object of the embodiments herein is to provide a wound dressing having a high durability.

Yet another object of the embodiments herein is to provide a wound dressing which is available easily, commercially and cost-effectively.

These and other objects and advantages of the embodiments herein will become readily apparent from the following detailed description taken in conjunction with the accompanying drawings.

### SUMMARY

The various embodiments herein provide a wound dressing film and a method of producing the same. According to one embodiment herein, the wound dressing film comprises a natural polymer, wherein the natural polymer is a chitosan; a biopolymer, wherein the biopolymers is selected from one or more elements from the group comprising a gelatin and a collagen; an artificial water absorber polymer or a synthetic polymer, wherein the polymer is selected from the group comprising of a hydrophilic material; and a paper or an aluminium foil.

According to one embodiment herein, the hydrophilic material is selected from one or more elements from the group comprising a polyvinyl alcohol (PVA) and a Polyvinylpyrrolidone, wherein the hydrophilic material is a polyvinyl alcohol (PVA).

According to one embodiment herein, a special method of producing a polymer wound dresser is provided wherein no drug is being used and wherein a combination of natural polymers like chitosan, gelatin, collagen and artificial polymers like polyvinyl alcohol is mixed with determinate percentages without using a drug and a thin film of the polymer combination is provided on a base using a manual film applicator at one face. A covering film or backing film made of paper or aluminum is laminated over the polymer film to form a wound dresser. The wound dresser is then dried at a room temperature and is further sterilized.

According to one embodiment herein, an absorbable wound dressing having a suitable bio-compatibility with the human and animal body is provided to act like a dam or a barrier against input and growth of microorganisms like virus that resulted in an infection and consequent delay in rehabilitation of ulcer. Further the wound dressing, may avoid an intense bleeding accompanied by wounds due its suitable structure.

According to one embodiment herein, the wound dressing has a quick drying nature to enable it to be used at home without needing a specialist for attaching it.

According to one embodiment herein, the wound dressing is easily separated from the injured surface after rehabilitation without causing skin damage and without leaving any substantial material residue, any inflammation or any special white color.

According to one embodiment herein, the chitosan has a variable molecular weight between low to high. The chitosan has a deacetylation degree between 82-99%. The weight percentage of a chitosan in the polymer combination is between 30-33%.

The weight percentage of the gelatin in the polymer combination is between 30-33%. The weight percentage of the collagen is 10-15%. The weight percentage of the polyvinyl alcohol (PVA) is 30-33%.

According to one embodiment herein, the wound dressing of the present invention does not contain any cross-linkers or crosslinking agents. Therefore, even with low molecular weight of chitosan, all amine (—NH2) groups are available and also no toxicity occurs due to absence of crosslinkers.

According to one embodiment herein, the change of (—NH2) groups to (NH3+) groups causes the preservation of acidic pH that enhances more antibacterial properties of chitosan and chitosan act as a polycation in such a condition.

According to one embodiment herein, the thickness of the wound dressing is 70-120 μm. The pore size of the wound dressing is 400 nm-1.4 nm.

According to one embodiment herein, the wound dressing is available as a square and a rectangular shaped film. The wound dressing is stretchable in nature.

According to one embodiment herein, the wound dressing is designed with a combination of synthetic and natural polymers to obtain a high biocompatibility and mechanical properties. The wound dressing prevents the wound from drying and scarring.

According to one embodiment herein, the wound dressing has an increased oxygen permeability, can be sterilized and can stanch bleeding and adherence. The wound dressing has good consistency with the skin anatomy and it can be absorbed by a human body. The wound dressing has an adequate resistance against tear, has stable properties in the applied range of temperature. The wound dressing is highly durable. The wound dressing is cost-effective and is available commercially.

According to one embodiment herein, a method for producing a wound dressing film comprises dissolving a preset amount of a chitosan and a preset amount of a collagen in a water solution containing a preset amount of a lactic acid with a preset volume to volume percentage to obtain a first solution; wherein the preset amount of lactic acid is 33 ml and the preset volume percentage of the lactic acid is 2% (v/v); dissolving a preset amount of a gelatin in a distilled water separately to obtain a second solution; dissolving a preset amount of polyvinyl alcohol (PVA) powder in a distilled water at a preset temperature of 90° C. at a preset time of 1 hour to obtain a third solution; cooling the third solution to a room temperature; mixing the obtained first solution, second solution and the third solution with a mixer to obtain a chitosan-PVA-gelatin-collagen polymer mixed solution; coating the obtained chitosan-PVA-gelatin-collagen-polymer mixed solution on an aluminum foil support with a preset thickness of 1500-2500 μm by using a manual film applicator or an automatic film applicator to obtain a film; drying the obtained film inside an oven at a preset temperature of 28-42° C.; sterilizing the film, wherein the sterilization is achieved by thermal and gamma radiation at a preset dose of 20 kGy; and packing the film for commercial use.

According to one embodiment herein, the wound dressing is a one layered or a two layered or a three layered. The two layered wound dressing comprises an adhesive layer and a backing layer. The three layered wound dressing comprises a release liner, an adhesive layer and a backing layer. The adhesive layer comprises a mixture of chitosan-polyvinyl alcohol-gelatin and collagen. The backing layer is selected from a paper or an aluminium foil, wherein the backing layer is of a thickness between 70-90 μm. The release liner comprises one or more elements selected from a group comprising of a siliconised polyethylene terephthalate, a silicon coated paper, a polypropylene, a polyester and a polyethylene.

According to one embodiment herein, a method of treatment of burns, injuries, surface and deep operations and other skin-related complications involves synthesizing a wound dressing film; placing the wound dressing film on an injured surface; keeping the wound dressing on the injured surface for a duration of 3-12 weeks; and separating the wound dressing from the injured surface easily without causing any skin damage, leaving a substantial material residue or a special white color after a removal of the wound dressing.

According to one embodiment herein, the wound dressing protects the injured skin and keeps the injured surface moist to speed up the healing process by absorbing exudates while activates immunocytes and inflammatory cells such as PMN, macro phage, fibroblasts and angio-endothelial cells. The wound dressing also has antibacterial properties.

According to one embodiment herein, the wound dressing absorbs bleeding by a rapid capillary action by drawing the exudates or infection within the middle layer of the dressing.

According to one embodiment herein, the wound dressing can be adhered to the human and animal body and it is suitable to be used in treatment of burning, cut injuries, as well as surface and deep operations.

According to one embodiment herein, the total time of treatment with the wound dressing in diabetic patients is 4-9 weeks, in Leishmaniasis patients it is 3-7 weeks and in burned patients it is 3-12 weeks.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a flow chart explaining the method of producing a wound dressing film, according to an embodiment herein.

FIG. 2 illustrates a general outline of a manual film applicator used in the method of producing the wound dressing film, according to an embodiment herein.

FIG. 3 illustrates a perspective view of an automatic film applicator used in the method of producing the wound dressing film, according to an embodiment herein.

FIG. 4 illustrates a side view of a one-layered wound dressing, two-layered wound dressing and three-layered wound dressing, according to an embodiment herein.

FIG. 5 illustrates an application of the wound dressing obtained by the method of producing the wound dressing film, according to an embodiment herein.

FIG. 6 illustrates a Scanning Electron Microscopy (SEM) micrograph of the surface of the wound dressing film with a pore size of 50 μm, acquired by a method of producing the wound dressing film, according to an embodiment herein.

FIG. 7 illustrates a Scanning Electron Microscopy (SEM) micrograph of the cross-section of the wound dressing film with a pore size of 10 μm, achieved by a method of producing the wound dressing film, according to an embodiment herein.

FIG. 8 illustrates a Scanning Electron Microscopy (SEM) micrograph of the cross-section of the wound dressing film with a pore size of 50 μm, achieved by a method of producing the wound dressing film, according to an embodiment herein.

FIG. 9 illustrates a Scanning Electron Microscopy (SEM) micrograph of the cross-section of the wound dressing film with a pore size of 10 μm, achieved by a method of producing the wound dressing film, according to an embodiment herein.

FIG. 10 illustrates a Scanning Electron Microscopy (SEM) micrograph of the cross-section of the wound dressing film with a pore size of 5 μm, achieved by a method of producing the wound dressing film, according to an embodiment herein.

Although specific features of the embodiments herein are shown in some drawings and not in others. This is done for convenience only as each feature may be combined with any or all of the other features in accordance with the embodiments herein.

### DETAILED DESCRIPTION OF THE EMBODIMENTS

In the following detailed description, a reference is made to the accompanying drawings that form a part hereof and in which the specific embodiments that may be practiced is shown by way of illustration. The embodiments herein are described in sufficient detail to enable those skilled in the art to practice the embodiments herein and it is to be understood that the logical, mechanical and other changes may be made without departing from the scope of the embodiments herein. The following detailed description is therefore not to be taken in a limiting sense.

The various embodiments herein provide a wound dressing film and a method of producing the same. According to an embodiment herein, a wound dressing film comprises a base polymer film and a backing layer laminated on the base polymer film. The base polymer film comprises a natural polymer, a biopolymer and an artificial water absorber polymer. The laminated backing layer is a paper. The laminated backing layer is an aluminium foil.

The natural polymer is a chitosan having a preset weight percentage. The preset weight percentage of a chitosan is between 30-33%.

The biopolymers comprises one or more elements selected from a group comprising of a gelatin having a preset weight percentage and a collagen having a preset weight percentage or a combination thereof The preset weight percentage of the gelatin is between 30-33% and the preset weight percentage of the collagen is 10-15%.

The artificial water absorber polymer is selected from a group comprising of a hydrophilic material having a preset weight percentage. The hydrophilic material is comprises one or more elements selected from a group comprising a polyvinyl alcohol (PVA) and a Polyvinylpyrrolidone. The hydrophilic material is a polyvinyl alcohol (PVA). The preset weight percentage of the polyvinyl alcohol (PVA) is 30-33%.

The thickness of the wound dressing is 70-120 μm. The pore size of the wound dressing is 400 nm-1.4 nm.

The wound dressing is a one layered wound dressing film or a two layered wound dressing film or a three layered wound dressing film. The two layered wound dressing film comprises of an adhesive layer and a backing layer. The three layered wound dressing film comprises of a release liner, an adhesive layer and a backing layer.

The adhesive layer comprises of a mixture of chitosan-polyvinyl alcohol-gelatin and collagen. The backing layer is a paper or an aluminium foil having a thickness between 70-90 μm. The release liner comprises one or more elements selected from a group comprising a polyethylene terephthalate siliconised, a silicon coated paper, a polypropylene, a polyester and a polyethylene or a combination thereof.

A method of producing a wound dressing film comprises dissolving a preset amount of a chitosan and a preset amount of a collagen in a water solution containing 33 ml of 2% (v/v) a lactic acid to obtain a first solution. A preset amount of a gelatin is dissolved in a distilled water separately to obtain a second solution. A preset amount of polyvinyl alcohol (PVA) powder is dissolved in a distilled water at a preset temperature of 90° C. at a preset time of 1 hour to obtain a third solution. The third solution cooled to a room temperature. The obtained first solution, second solution and the third solution are mixed using a mixer to obtain a chitosan-PVA-gelatin-collagen polymer mixed solution. The obtained chitosan-PVA-gelatin-collagen-polymer mixed solution is coated on an aluminum foil support with a preset thickness of 1500-2500 μm by using a manual film applicator or an automatic film applicator to obtain a wound dressing film. The obtained wound dressing film is dried inside an oven at a preset temperature of 28-42° C. Then the dried wound dressing film is sterilized. The dried wound dressing film is sterilized by thermal and gamma radiation at a preset dose of 20 kGy. The sterilized film is packed for commercial use.

According to one embodiment herein, a wound dressing film comprises a natural chitosan and derivates of polymer and other biopolymers such as gelatin and collagen and synthetic polymers like polyvinyl alcohol and polyvinylpyrrolidone and a paper or an aluminium foil.

Chitosan enhances the functions of inflammatory cells such as polymorphonuclear leukocytes, macrophages and fibroblasts thereby promoting granulation and organization. It is biocompatible, biodegradable, hemostatic, anti-infective and, antibacterial activity against a broad spectrum of bacteria. The weight percentage of chitosan and its derivatives in the wound dressing is between 30-33%.

Gelatin impairs haemostasis and blood coagulation and influence platelet aggregation after cardiac surgery. The weight percentage of gelatin in the wound dressing is between 30-33%.

Collagen is the most promising skin substitute or wound dressing biomaterial, due to minimal inflammation, cytotoxicity and its property to promote cellular growth. Collagen also plays a critical role in all phases of wound healing (hemostasis, inflammation, proliferation, and remodelling). The weight percentage of collagen in the wound dressing is between 10-15%.

Polyvinyl alcohol (PVA) provides the mechanical strength of wound dressing. The weight percentage of polyvinyl alcohol (PVA) in the wound dressing is between 30-33%.

According to one embodiment herein, the wound dressing film does not contain any cross-linkers or cross-linking agents. Therefore all amine (—NH2) groups are available even with low molecular weight of chitosan and the wound dressing film does not have any toxicity due to the absence of cross-linking agents.

According to one embodiment herein, the change of (—NH2) groups to (NH3+) groups causes the preservation of acidic pH that enhances more antibacterial properties of chitosan and the chitosan act as a polycation in such a condition.

According to one embodiment herein, the wound dressing can be one layered or two layered or three layered without degrading the efficacy of the dresser. A two layered wound dressing comprises of an adhesive layer and a backing layer. A three layered wound dressing comprises of a release liner, an adhesive layer and a backing layer.

According to one embodiment herein, a wound dressing composition mixture of chitosan-polyvinyl alcohol-gelatin and collagen acts as an adhesive layer. A paper or an aluminium foil acts as a backing layer having a thickness of 70-90 μm that may be coated with the wound dressing composition and is separated from wound dressing film after film is dried. A polyethylene terephthalate siliconised acts as a release liner. The release liner may also be selected from a group that comprises a silicon coated paper or any polymer such as a polypropylene, a polyester and a polyethylene.

According to an embodiment herein, the chitosan with low, medium and high molecular weight has a deacetylation degree of about 82%. Also the chitosan with low molecular weight has a deacetylation degree of about 98-99%.

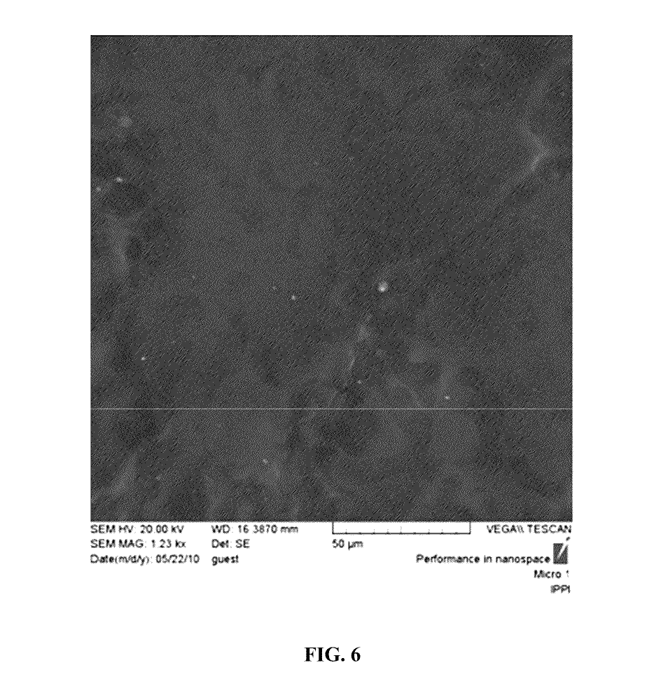

According to one embodiment herein, the wound dressing is of 70-120 μm thickness. From the scanning electron microscopy (SEM) images (FIG. 6-FIG. 10), it is observed that the outer layer is dense and the internal layer is porous. In FIG. 6 a smooth and a dense skin layer is observed on the wound dressing film. The pore size of the wound dressing is 400 nm to 1.4 nm having irregular shape. The wound dressing is of a square or a rectangular in shape. The wound dressing is stretchable.

According to one embodiment herein, the polymer film prepared by the method provided in the present invention is placed between wound and environment facilitating transfer of water vapor for the treatment of injury.

According to one embodiment herein, the wound dressing protects the injured skin and keeps it appropriately moist to speed up the healing process by absorbing exudates while activating the immunocytes and the inflammatory cells such as PMN, macrophage, fibroblasts and angio-endothelial cells. It also has antibacterial properties.

The dressing has a rapid capillary action that draws exudates or infection within the middle layer of the dressing.

According to one embodiment herein, a wound dressing has a remarkable bio-compatibility and absorbs the exudates. It has excellent mechanical and antibacterial characteristics. It can be absorbed by a human body, can be sterilized and may be used with/without a backing layer or a release liner and is suitable in terms of price.

According to one embodiment herein, the absorbable wound dressing film has a suitable bio-compatibility with the human and animal body. It can act like a dam or barrier against input and growth of microorganisms like virus that result in infection and causes consequent delay in the rehabilitation of ulcer.

According to one embodiment herein, the wound dressing film has a suitable structure that may avoid bleeding in wounds accompanied by an intense bleeding such as in cases of operations in which the patient suffers from intense hemorrhage.

One of the other characteristics of the wound dresser of the embodiments herein is its quick drying nature that enables it to be used easily at home without needing specialist for attaching it.

According to one embodiment herein, the wound dressing film is easily separated from the injured surface after treatment without causing skin damage and without leaving any substantial material residue, inflammation or a special white color after removal of the wound dresser.

The wound dressing film can be adhered to the human and animal body and it is suitable to be used in treatment of burning, cut injuries, as well as surface and deep operations. The total time of treatment with the wound dressing in diabetic patients is 4-9 weeks, in Leishmaniasis patients it is 3-7 weeks and in burned patients it is 3-12 weeks.

### EXPERIMENTAL DATA

To determine the biocompatibility and cytocompatibility of the composite film containing the wound dresser composition, in vitro fibroblast culture (Fibroblast were isolated from human skin) was performed using the composite film and the normal culture medium. The samples with a diameter of 2 cm was sterilized by temperature (T=120, t=1 h) and then washed with phosphate buffer saline (pH=7.4) four times. The sample was then placed in a tissue culture polystyrene plate. The results show that the composite film presented in this invention does not have any cytotoxicity and had good in vitro biocompatibility.

Hence the composite film can be safely used as good wound dressing.

### Water-Uptake Capacity

After providing films, we submerge them inside water and using the following formula calculate water absorption rate by them:

$$Sw ( \% ) = \frac{Ws - Wd}{Wd} = 100$$

In this formula, Ws is the weight of wet film and Wd is the weight of dry film. Results in this connection represent higher absorbability of these films that depending on the type of sterilization it may absorb (30-250) % of water that these digits may represent higher absorption rate of infection in the time of using such films as a wound dressing.

### Mechanical Characteristics of the Achieved Samples

Using “SANTAM, STM-20, IRAN” System, tensile strength and elongation at break was studied. For this task, samples were cut with dimensions of 10×1.5 cm and their diameter was also analyzed. Tensile strength of these samples was achieved being equal to 42N/mm2 and elongation at break was acquired being equal to 130%.

### Water Vapor Permeability

One of the main parameters of wound dressing film is transmission of body liquid or wound exudates. The water vapor permeability of the films was determined using the ASTM method E 96-95 (ASTM, 1995). The films were fixed on the top of test cells containing a desiccant such as a silica gel. Test cells were placed in a chamber with controlled temperature at 25° C. and relative humidity of 75% RH.

Permeation cells were weighed before and after five days of incubation, and then the acquired weight was used to calculate the water vapor permeability.

Water vapor permeability of native chitosan film was 0.54 ng m/m2 s pa. Incorporation of other polymer into chitosan films increased the WVP (WVP=0.70 ng m/m2 s pa).

These results suggest that the enhancement of the mechanical properties and water vapor permeability of the composite films was due to intermolecular interaction by hydrogen bonding.

### Cytotoxicity Evaluation

To determine the biocompatibility and cytocompatibility of the composite film containing the wound dresser, in vitro fibroblast culture (Fibroblast were isolated from human skin) was performed using the composite film and the normal culture medium. The samples with a diameter of 2 cm was sterilized by temperature (T=120, t=1 h) and then was washed with phosphate buffer saline (pH=7.4) four times. The sample was then placed in a tissue culture polystyrene plate.

The results show that the composite film in the present invention does not have any cytotoxicity and had good in vitro biocompatibility.

Hence the composite film can be safely used as good wound dressing.

The embodiments herein are related to a non-drug wound dressing and a method of producing the same.

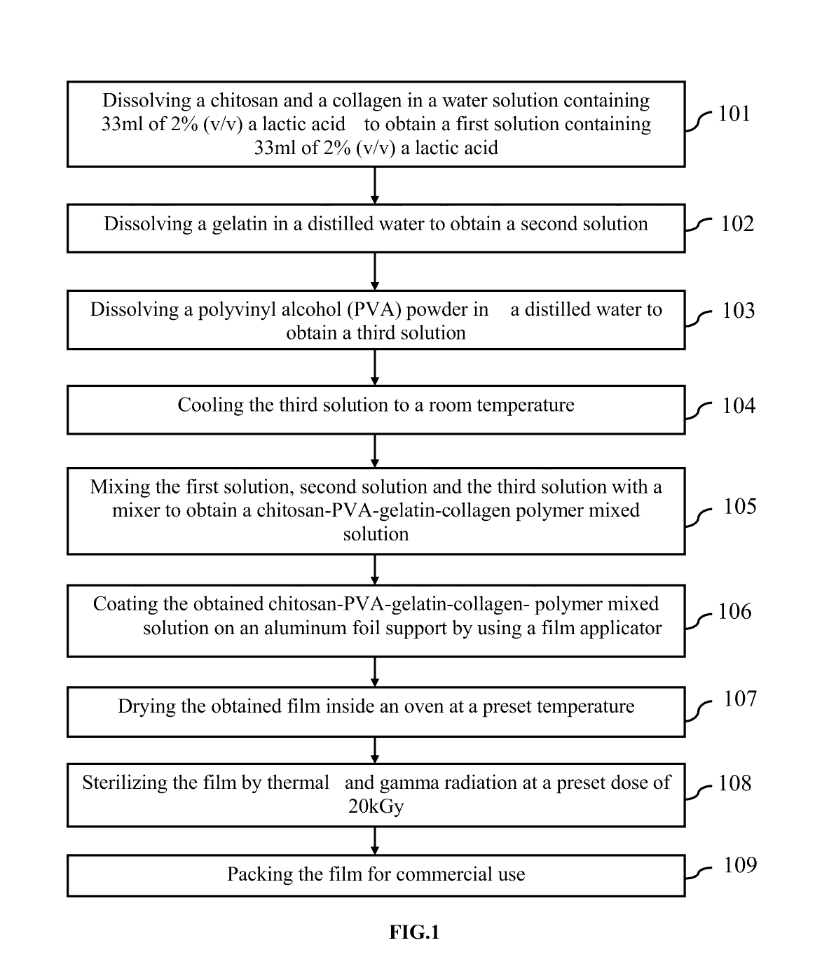

FIG. 1 illustrates a flow chart explaining a method of producing a wound dressing film according to one embodiment herein. With respect to FIG. 1, the method of producing a wound dressing film, involves dissolving a preset amount of a chitosan and a preset amount of a collagen in a water solution containing 33 ml of 2% (v/v) a lactic acid to obtain a first solution (101). A preset amount of a gelatin is dissolved in a distilled water separately to obtain a second solution (102). A preset amount of polyvinyl alcohol (PVA) powder is dissolved in a distilled water at a preset temperature of 90° C. at a preset time of 1 hour to obtain a third solution (103). The third solution is cooled to a room temperature (104). The obtained first solution, second solution and the third solution are mixed using a mixer to obtain a chitosan-PVA-gelatin-collagen polymer mixed solution (105). The obtained chitosan-PVA-gelatin-collagen-polymer mixed solution is coated on an aluminum foil support with a preset thickness of 1500-2500 μm by using a manual film applicator 103TSS Tohid Sanat Sepahan or an automatic film applicator 1031TSS Tohid Sanat Sepahan, (Iran) to obtain a wound dressing film (106). The obtained wound dressing film is dried inside an oven at a preset temperature of 28-42° C. (107). The dried wound dressing film is sterilized by a thermal and gamma radiation at a preset dose of 20 kGy (108). The sterilized film is packing for a commercial use (109).

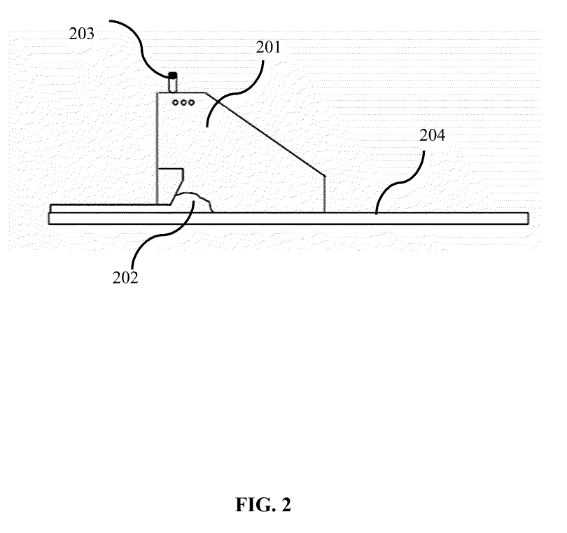

FIG. 2 illustrates a general outline of the manual film applicator used in the method of producing the wound dressing film, according to an embodiment herein. With respect to FIG. 2, the manual film applicator 201 is used to coat the polymer mixture 202 with a considered thickness of 1500-2500 μm on a backing layer 204, wherein the backing layer 204 is selected from a paper or an aluminium foil having a thickness between 70-90 μm. The thickness of the wound dressing film is controlled by a micrometer 203 of the manual film applicator 201.

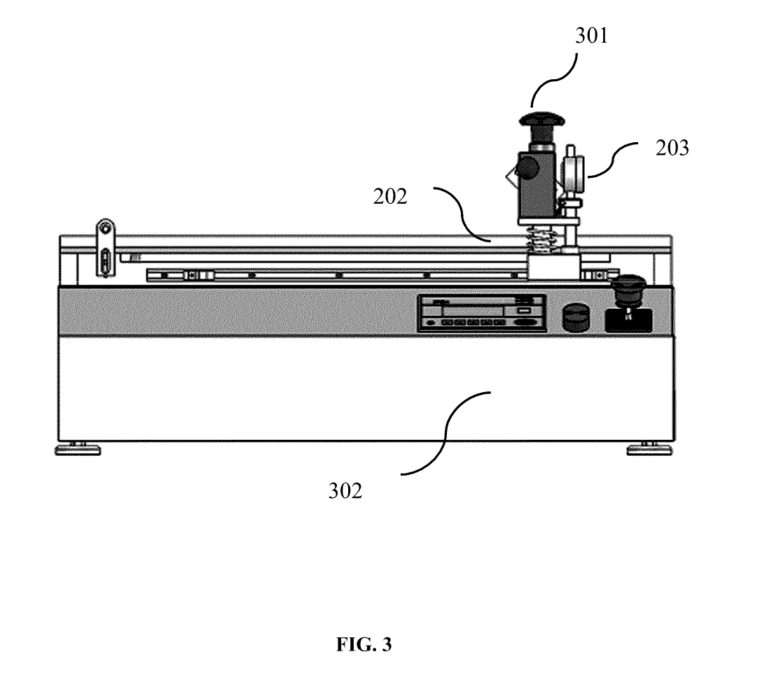

FIG. 3 illustrates a perspective view of the automatic film applicator used in the method of producing the wound dressing film, according to an embodiment herein. With respect to FIG. 3, the automatic film applicator 301 is used for the coating of the wound dressing composition 202 on the backing layer 204, wherein the backing layer is selected from a paper or an aluminium foil having a thickness between 70-90 μm with a micrometer 203 and digital control panel 302 for controlling the thickness and speed of the coating.

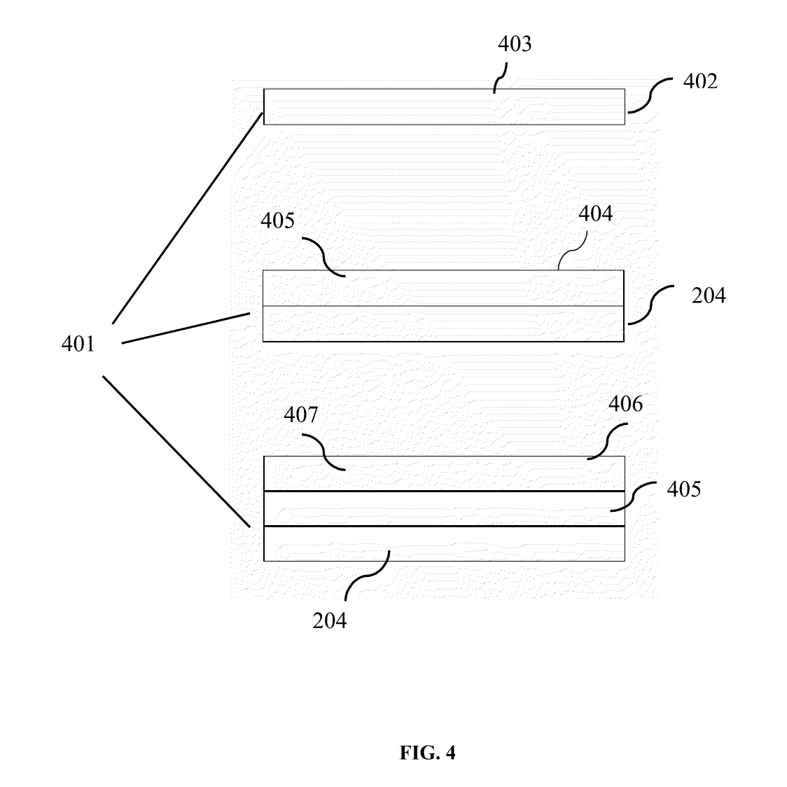

FIG. 4 illustrates the structural alignment of the one-layered wound dressing, two-layered wound dressing and three-layered wound dressing so achieved by the method of producing the wound dressing film, according to an embodiment herein. With respect to FIG. 4, the three various type of the wound dressing film 401 are produced by a method according to one embodiment herein.

A one-layered laminated wound dressing film 402 is a non drug based polymer film 403. The two-layered wound dressing film 404 has an adhesive layer 405 and a backing layer 204. The adhesive layer 405 comprises of a mixture of chitosan-polyvinyl alcohol-gelatin and collagen. The backing layer 204 is selected from a paper or an aluminium foil, having a thickness between 70-90 μm. The three-layered wound dressing film 406 has a release liner 407, an adhesive layer 405 and a backing layer 204. The release liner 407 comprises one or more elements selected from a group comprising a siliconised polyethylene terephthalate, a silicon coated paper, a polypropylene, a polyester and a polyethylene.

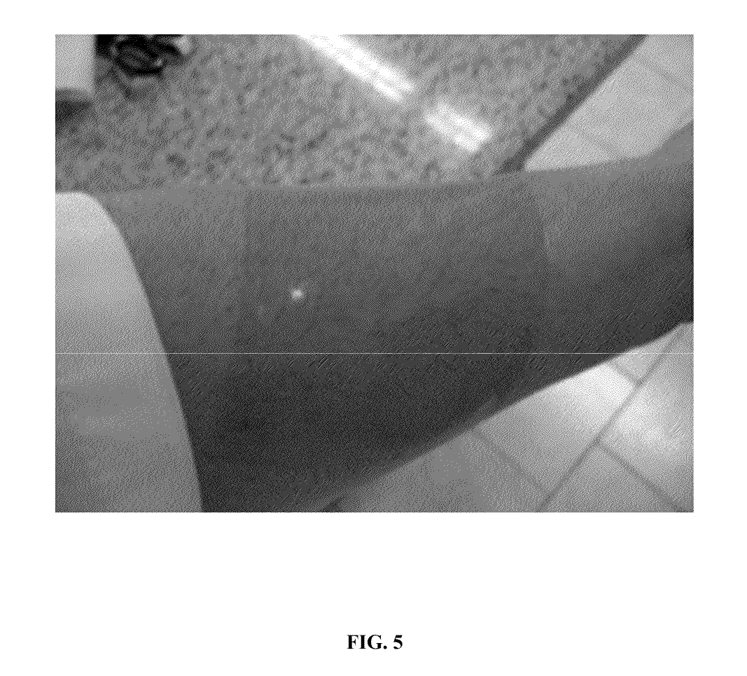

FIG. 5 illustrates an application of the wound dressing obtained by the method of producing the wound dressing film, according to an embodiment herein. With respect to FIG. 5, a synthesized wound dressing film is placed on the injured surface. The wound dressing film is made to rest on the injured surface for duration of 3-12 weeks. Then the wound dressing is separated from the injured surface easily without causing any skin damage and leaving substantial material residue or a special white color after removal of it.

FIG. 6 illustrates a Scanning Electron Microscopy (SEM) micrograph of the surface of the wound dressing film with a pore size of 50 μm, acquired by the method of producing the wound dressing film, according to an embodiment herein.

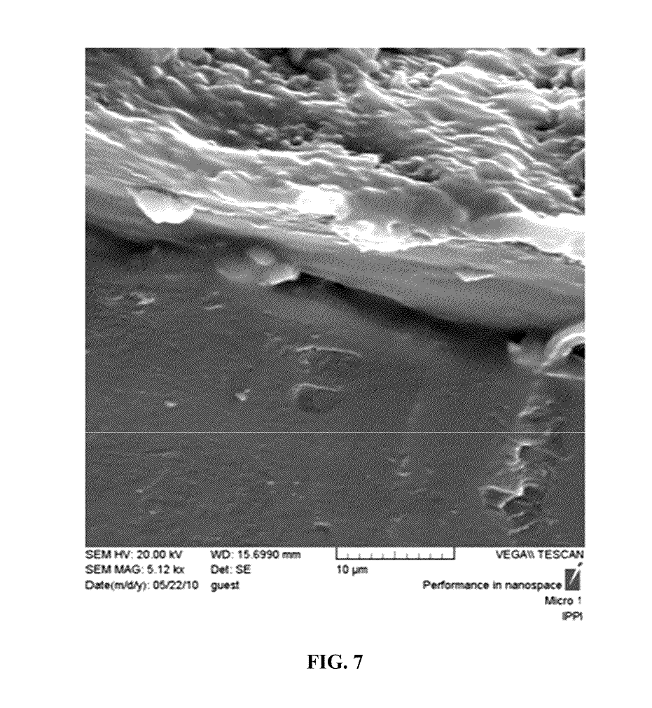

FIG. 7 illustrates a Scanning Electron Microscopy (SEM) micrograph of the cross-section of the wound dressing film with a pore size of 10 μm, achieved by the method of producing the wound dressing film, according to an embodiment herein.

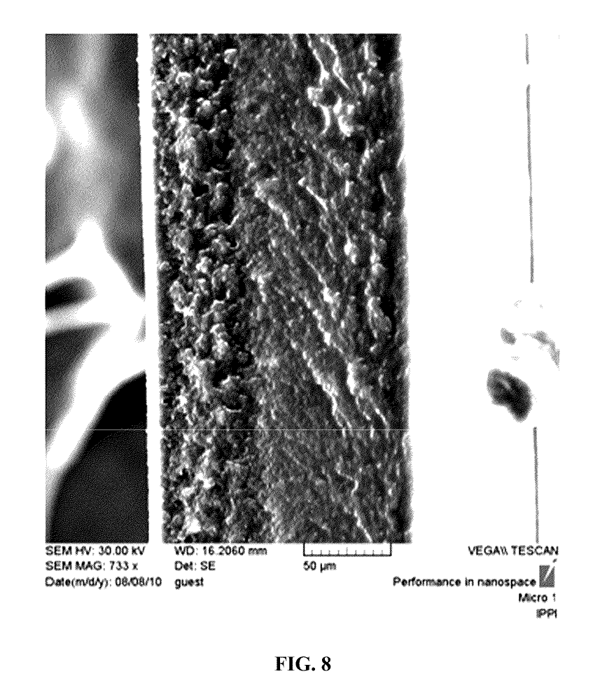

FIG. 8 illustrates a Scanning Electron Microscopy (SEM) micrograph of the cross-section of the wound dressing film with a pore size of 50 μm, achieved by the method of producing the wound dressing film, according to an embodiment herein.

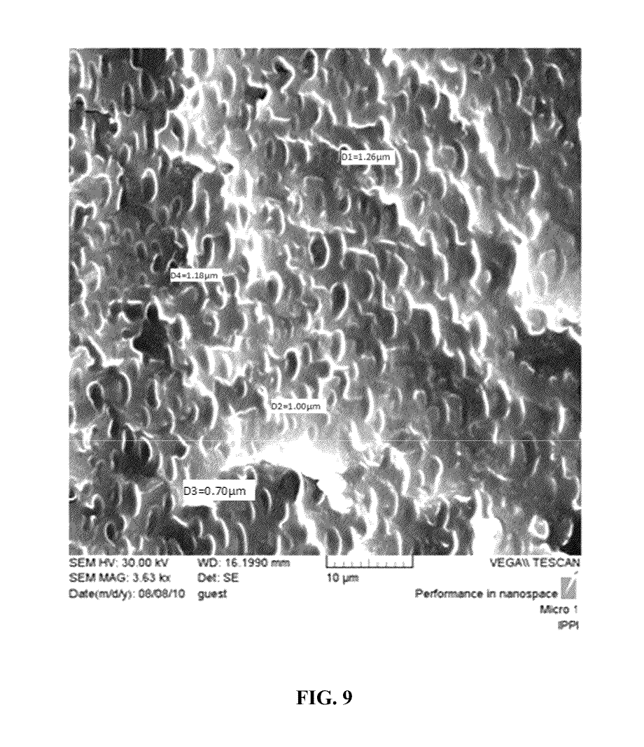

FIG. 9 illustrates a Scanning Electron Microscopy (SEM) micrograph of the cross-section of the wound dressing film with a pore size of 10 μm, achieved by the method of producing the wound dressing film, according to an embodiment herein.

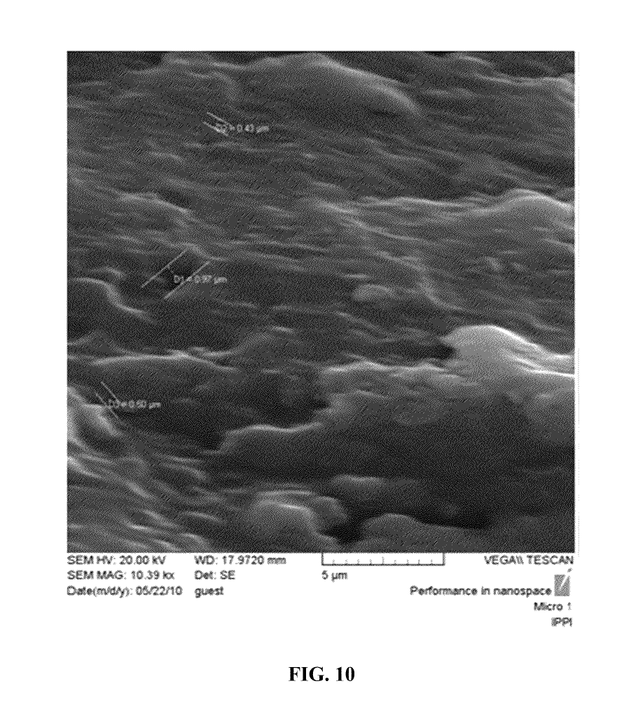

FIG. 10 illustrates a Scanning Electron Microscopy (SEM) micrograph of the cross-section of the wound dressing film with a pore size of 5 μm, achieved by the method of producing the wound dressing film, according to an embodiment herein.

It is noted that the disclosed arrangements are only illustrative of the application of the principles of the embodiments herein. Many modifications may be contrived in the structures illustrated herein without departing from the spirit and scope of the embodiments herein. The disclosed embodiments herein are to be considered in all respects only as illustrative and not restrictive. The scope of the embodiments herein is also indicated by the appended claim.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the embodiments herein with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A wound dressing film consisting of:
a base polymer film;
a backing layer laminated on the base polymer film;
wherein the base polymer film includes a natural polymer, a biopolymer and an artificial water absorber polymer, wherein the natural polymer is a chitosan having a preset weight percentage, wherein the biopolymer comprises one or more element selected from a group comprising of a gelatin having a preset weight percentage and a collagen having a preset weight percentage and a combination thereof; and wherein the artificial water absorber polymer is selected from a group comprising of a hydrophilic material having a preset weight percentage.

2. The film according to claim 1, wherein the laminated backing layer is a paper with a thickness of 70-90 μm.

3. The film according to claim 1, wherein the laminated backing layer is an aluminium foil with a thickness of 70-90 μm.

4. The film according to claim 1, wherein the preset weight percentage of a chitosan is within 30-33%.

5. The film according to claim 1, wherein the preset weight percentage of the gelatin is within 30-33%.

6. The film according to claim 1, wherein the preset weight percentage of the collagen is 10-15%.

7. The film according to claim 1, wherein the hydrophilic material comprises one or more elements selected from a group comprising a polyvinyl alcohol (PVA) and a Polyvinylpyrrolidone.

8. The film according to claim 1, wherein the hydrophilic material is a polyvinyl alcohol (PVA).

9. The film according to claim 1, wherein the preset weight percentage of the polyvinyl alcohol (PVA) is 30-33%.

10. The film according to claim 1, wherein the thickness of the wound dressing film is 70-120 μm.

11. The film according to claim 1, wherein the wound dressing film has a pore with a size of 400 nm-1.4 nm.

12. The film according to claim 1, wherein the wound dressing film is a one layered wound dressing film.

13. The film according to claim 1, wherein the wound dressing is a two layered wound dressing film.

14. The film according to claim 1, wherein the wound dressing film is a three layered wound dressing film.

15. The film according to claim 1, wherein the two layered wound dressing film comprises an adhesive layer and a backing layer.

16. The film according to claim 1, wherein the three layered wound dressing film comprises a release liner, the adhesive layer and the backing layer.

17. The film according to claim 1, wherein the adhesive layer comprises a mixture of chitosan-polyvinyl alcohol-gelatin and collagen.

18. The film according to claim 1, wherein the release liner comprises one or more elements selected from a group comprising a polyethylene terephthalate siliconised, a silicon coated paper, a polypropylene, a polyester and a polyethylene or a combination thereof

19. The film according to claim 1, wherein the backing layer is a paper or aluminium foil having a thickness between 70-90 μm.

20. A method of producing a wound dressing film, wherein the method comprises:
dissolving a preset amount of a chitosan and a preset amount of a collagen in a water solution to obtain a first solution; and wherein the water solution includes a preset amount of a lactic acid with a preset volume to volume percentage; wherein the preset amount of water is 33 ml and wherein the volume to volume (v/v) percentage is 2%;
dissolving a preset amount of a gelatin in a distilled water separately to obtain a second solution;
dissolving a preset amount of polyvinyl alcohol (PVA) powder in a distilled water at a preset temperature of 90° C. for a preset time of 1 hour to obtain a third solution;
cooling the third solution to a room temperature;
mixing the first solution, the second solution and the third solution with a mixer to obtain a chitosan-PVA-gelatin-collagen polymer mixed solution;
coating the chitosan-PVA-gelatin-collagen-polymer mixed solution on an aluminium foil support with a preset thickness of 1500-2500 μm by using a manual film applicator or an automatic film applicator to obtain a polymer film;
drying the polymer film inside an oven at a preset temperature of 28-42° C.;
sterilizing the dried polymer film, wherein the sterilization is achieved by a thermal radiation and a gamma radiation at a preset dose of 20 kGy; and
packing the sterilized polymer film for a commercial use.
